(19) United States
(12) Patent Application Publication
(10) Pub. No.: US 2007/0036766 A1
(43) Pub. Date: Feb. 15, 2007

(54) Tissue graft composition comprising autologous bone marrow and purified autologous thrombin

(76) Inventors: Sherwin Kevy, Brookline, MA (US); May Jacobson, Sudbury, MA (US); James Ellsworth, Marshfield, MA (US); Kevin Benoit, Duxbury, MA (US)

Correspondence Address: HESLIN ROTHENBERG FARLEY & MESITI PC, 5 COLUMBIA CIRCLE, ALBANY, NY, 12203, US

(21) Appl. No.: 11/200,535

(22) Filed: Aug. 9, 2005

(51) Int. Cl. A61K 35/14; C12N 5/08

(52) U.S. Cl. 424/93.7; 623/919; 435/378; 435/372

(57) ABSTRACT

A method for the preparation of an autologous tissue graft composition is disclosed. Bone marrow is harvested from the patient. In one embodiment, the nucleated cells of the bone marrow are subsequently concentrated. Autologous thrombin is purified from a volume of whole blood also taken from the patient. The bone marrow aspirate or bone marrow concentrate is then combined with the purified autologous thrombin to form a coagulated tissue graft material that may be used alone or in conjunction with other graft materials.

### FIELD OF THE INVENTION

The present invention relates to a point-of-care method for the preparation of a tissue graft/wound healing composition capable of enhancing tissue regeneration wherein the composition comprises a bone marrow aspirate or bone marrow concentrate and purified autologous thrombin.

### BACKGROUND OF THE INVENTION

Bone and soft tissue grafting is a common surgical procedure to achieve wound closure. An example of bone grafting is the fusion of bone tissue for the repair of degenerative, traumatic, oncologic or infectious conditions. An example of soft tissue grafting is the application of autogenous skin recovered from a donor site and applied to a soft tissue injury in order to facilitate healing. The autogenous skin graft creates a biocompatible and bioactive wound covering and wound environment that may enhance wound healing.

There are three functional characteristics of a graft material that make it effective in facilitating repair. For a bone graft to be osteoconductive, it must provide a scaffold upon which new bone tissue can grow. Osteoconductive bone graft materials typically include synthetic matrices, autogenous bone and matrices of cadaveric origin.

Bone graft materials that are osteoinductive include factors, among them bone morphogenetic proteins, which recruit progenitor cells to the graft site and subsequently induce growth and differentiation of the progenitor cells. Osteogenic bone graft materials generally contain the precursor cells that ultimately differentiate to form new bone tissue.

To date, the gold standard for graft material is autogenous, that is, tissue taken from the patient, because it possesses all three functional characteristics described above and is inherently non-immunological. Due to the high incidence of donor site morbidity associated with autogenous grafts, however, there has been a great deal of interest in developing other graft substitutes with reduced morbidity.

In addition to being the repository of precursor cells needed for hematopoiesis, bone marrow also contains a population of mesenchymal stem cells (MSC) which are capable of differentiating into different mesodermal tissues, including bone, muscle, tendon and fat.

Bone marrow has been shown to enhance the rate of bone formation in animal long bone defect models and enhance the rate of healing of soft tissue defects. Graft materials consisting of bone marrow alone or in conjunction with another tissue graft material, have been investigated as potential tissue graft substitutes, with mixed results. Scott et al., for example, reported that coralline hydroxyapatite supplemented with bone marrow was not an acceptable bone graft substitute for posterolateral spine fusion (Scott et al., The Use of Coralline Hydroxyapatite With Bone Marrow, Autogenous Bone Graft, or Osteoinductive Bone Protein Extract for Posterolateral Lumbar Spine Fusion, Spine 24:320-327, 1999.) Muschler et al., on the other hand, demonstrated that selected implantable matrices that have been enriched with bone marrow-derived osteoblast progenitor cells improve the outcome of bone grafting (Muschler et al., Spine Fusion Using Cell Matrix Composites Enriched in Bone Marrow-Derived Cells, Clinical Orthopaedics and Related Research, 407:102-118, 2003. Badiavas and Falanga (Treatment of chronic wounds with bone marrow-derived cells. Arch. Dermatol. 139(4): 510-516 2003) demonstrated that chronic non-healing wounds treated with autogenous bone marrow can be encouraged to heal.

What is needed is a tissue graft material that has all the advantages of autogenous tissue, which is readily available and can be easily prepared and manipulated at or near point-of-care and returned and applied to a patient within the time-frame of the surgical procedure.

### SUMMARY OF THE INVENTION

The method of the present invention provides for the preparation of an autologous tissue graft material derived from bone marrow and peripheral whole blood harvested from the patient. The bone marrow can be concentrated to yield an increased number of nucleated cells such as osteogenic and hematopoietic progenitors. Purified autologous thrombin is purified from whole blood and combined with the bone marrow or bone marrow cell concentrate to generate a graft material that may be used alone, eliminating the need for harvesting autogenous skin or bone, or in combination with supplemental graft materials including autograft, allograft, xenograft or a synthetic graft composition, prior to application to the graft site.

In one aspect, therefore, the invention relates to a tissue graft composition comprising autologous bone marrow or a concentrate of bone marrow-derived cells and purified autologous thrombin. The composition may further comprise a graft material selected from autograft, allograft, xenograft and a synthetic graft material.

In a related aspect, the invention relates to a composition comprising autologous bone marrow or bone marrow concentrate, purified autologous thrombin and a platelet concentrate or platelet rich plasma. This composition may further comprise a graft material selected from autograft bone, allograft, xenograft and a synthetic graft material.

In yet another aspect, the invention relates to a method for the preparation of a tissue graft composition comprising a bone marrow concentrate and purified autologous thrombin. The method comprises:
a) obtaining a volume of anticoagulated bone marrow from a patient;
b) obtaining a concentrate of said bone marrow;
c) mixing the cell concentrate with purified autologous thrombin to obtain the tissue graft composition.

In a related aspect, the invention relates to a method for the preparation of a tissue graft composition wherein the method comprises the further step of mixing the graft composition with a supplemental graft material. Examples of suitable graft materials include allograft, autograft, mineralized or demineralized bone, hydroxyapetite or other synthetic matrix material.

### DETAILED DESCRIPTION OF THE INVENTION

All patents, applications, publications, or other references that are listed herein are hereby incorporated by reference. In the description that follows, certain conventions will be followed as regards the use of terminology:
ACD acid-citrate-dextrose
CaCl₂ calcium chloride
CPD citrate-phosphate-dextrose
EDTA ethylenediamine tetraacetic acid
ETOH ethanol, ethyl alcohol
PEG polyethylene glycol
BMC bone marrow concentrate
PPP platelet-poor plasma
PRP platelet-rich plasma
PC platelet concentrate

The term “tissue graft material” or “tissue graft composition” refers to a composition applied to a wound site that is the result of injury, disease or surgery and includes sites in bone and cartilage as well as soft tissue. The tissue graft material is used to promote healing and tissue regeneration and can be used therefore, as a wound healing composition.

The term “anticoagulant” refers to a substance capable of preventing whole blood from clotting. Any anticoagulant capable of inhibiting coagulation of a whole blood or bone marrow specimen is suitable for use in practicing the method of the present invention; examples include, without limitation, ethylenediamine tetraacetic acid (EDTA), heparin and preferably, citrate-based anticoagulants, such as acid-citrate-dextrose (ACD) and citrate-phosphate-dextrose (CPD).

The term “bone marrow concentrate,” or “concentrate of bone marrow-derived cells” refers to an enriched population of bone-marrow-derived nucleated cells without regard to selection of a subset of nucleated cells. In a preferred embodiment, the bone marrow is concentrated by centrifugation of an anticoagulated bone marrow aspirate to generate a product in which nucleated cells and platelets are concentrated and red blood cells and plasma are consequently reduced.

The term “point-of-care,” as used herein to describe the graft material preparation method of the invention, refers to diagnostic or treatment-related procedures performed at or near the site of patient care. Point-of-care procedures are generally associated with enhanced efficiency and improved outcome.

The tissue graft material of the present invention derives its benefit from its autologous nature, its rapid preparation and its flexibility to be able to enhance tissue regeneration alone or in combination with other graft materials. The tissue graft material of the present invention combines an autologous bone marrow aspirate or concentrate with a purified autologous thrombin preparation.

The bone marrow component is the source of cellular components, both differentiated and undifferentiated.

The benefits of this particular combination derive from the flexibility associated with first obtaining an anticoagulated autologous bone marrow aspirate, which can then be manipulated, for example, concentrated by reduction of the red blood cell and plasma components and then coagulating the bone marrow preparation in a controlled fashion by the addition of autologous thrombin to the bone marrow aspirate or concentrate.

Coagulation of the bone marrow preparation is critical to hemostasis and the stabilization of the graft within the site. Coagulation of the bone marrow also improves handling characteristics and helps the graft adhere to the wound. Coagulation of the bone marrow after processing ensures delivery to the graft site of an optimal amount of the cellular and protein components necessary for improved tissue regeneration. Being able to control the coagulation event, therefore, is an important feature of the present invention.

Preparation of Graft Material

Bone marrow from the patient is harvested in accordance with accepted medical practice, generally, from a site in the iliac crest, tibia, humerus etc. Bone marrow is collected into a syringe (multiple syringes may be required, depending on the amount of bone marrow material needed for the graft) containing an anticoagulant, such as heparin, ACD or CPD to prevent clotting. Optimally, the volume of anticoagulant required is approximately 15-20% of the bone marrow volume, although other anticoagulant volumes may also be effective. Alternatively, the bone marrow is collected in a syringe not containing anticoagulant but is subsequently expressed into a suitable container containing the appropriate amount of anticoagulant for further processing.

In one embodiment, the anticoagulated bone marrow is then centrifuged at, for example, 1,000×g for 10 minutes to obtain fractionation of the various bone marrow/whole blood components into discrete regions of the centrifugation vessel. Plasma and red blood cells (RBC) are discarded and the buffy coat, which contains the concentrated bone marrow-derived nucleated cells (BMC) and platelets, is recovered.

Bone marrow components may be fractionated using standard centrifugation techniques known to those of skill in the art. In one embodiment, a bone marrow concentrate is obtained by placing the bone marrow aspirate in a sterile processing disposable compatible with an automated centrifugation system, such as the SMARTPREP® system (Harvest Technologies Corp, Plymouth, Mass.). This system, as described in U.S. Pat. No. 5,707,331, consists of an automated microprocessor controlled centrifuge with decanting capability and a swinging bucket designed to allow for rapid automatic separation of plasma and platelets from a sample of whole blood. The system uses a dual chambered processing disposable container of 20 ml and 60 ml volume capable of processing any volume of blood or bone marrow from 18 ml to 60 ml. A unique feature of the disposable is a floating shelf of a specific gravity that rises during the initial centrifugation step and is capable of separating red blood cells from other blood/marrow components.

The anticoagulated bone marrow is first placed in one chamber of the container. The centrifuge is then operated to cause the red blood cells to sediment to the bottom of the chamber. Centrifugation is stopped causing the RBC-reduced bone marrow to decant to a second chamber. The container is then centrifuged a second time resulting in a density-dependent separation of the nucleated cells of the bone marrow from the plasma component. Most of the plasma is removed. The bone marrow concentrate is then harvested and resuspended to a concentration of approximately 2 to 4 times baseline levels.

The system described above was used to concentrate canine bone marrow. As shown in Table 1, centrifugation resulted in a 5-6 fold concentration of the leukocytic population which was paralleled by the concentration of myeloblasts (mitotic myeloid). Flow cytometric analysis utilizing antibodies against CD34, CD44 and CD45 confirmed that this increase was also seen in the mesenchymal stem cell population.

TABLE 1

| | | | Mitotic | Erythroid |
|---|---|---|---|---|
| Sample | WBC | M:E | Myeloid | Precursors |
| Preparation | (×103/μl) | Ratio | % | 1% |
| Sample #1 | | | | |
| Preconcentration | 61.6 | 3.17:1 | 6 | 24 |
| Post concentration | 346.2 | 1.63:1 | 25 | 38 |
| Sample #2 | | | | |
| Preconcentration | 59.4 | 1.86:1 | 0.1 | 35 |
| Post concentration | 459.6 | 2.45:1 | 23 | 29 |

In yet another embodiment, a bone marrow concentrate is obtained using the method and apparatus described in U.S. application serial number PCT/US04/15654. Briefly, the bone marrow is aspirated into a syringe which acts as the processing disposable. The anticoagulated bone marrow preparation is then centrifuged at, for example, 1,000×g for 10 mins. To facilitate recovery of the desired nucleated cell fraction, the syringe, like the processing disposable described above contains a density disk assembly which floats within the syringe such that the assembly encompasses the fractionated nucleated cells. The position of the disc is determined by the patient's hematocrit.

Subsequently, the bone marrow concentrate is combined with purified autologous thrombin to form the bone graft material of the invention.

Preparation of Autologous Thrombin

Prior to or concurrently with the preparation of the bone marrow concentrate, autologous thrombin is purified from an aliquot of whole blood taken from the patient. The methodology for the isolation of a purified autologous thrombin preparation is described in co-pending U.S. application Ser. No. 10/765,694. Briefly, purified autologous thrombin is obtained from a whole blood sample taken from the patient. The method comprises the steps of a) obtaining a volume of anticoagulated whole blood from the patient; b) mixing the anticoagulated whole blood with a precipitating agent; c) incubating the mixture of b) for a time sufficient for precipitation of cellular and specific plasma components to occur; d) separating the precipitate obtained in c) from the supernatant (usually by centrifugation and/or filtration); and e) recovering the supernatant wherein the supernatant contains purified autologous thrombin.

In one embodiment, a small volume of anticoagulated whole blood is obtained by drawing blood from the patient into a blood collection tube or syringe which contains an anticoagulant, for example, acid-citrate-dextrose. After thorough but gentle mixing, the anticoagulated whole blood is transferred to a glass or plastic tube containing a precipitating agent, such as ethanol, and is mixed with the anticoagulated whole blood. The resulting mixture is incubated at room temperature for a period of time sufficient for precipitation of the cellular and specific plasma components of the blood to occur, about 20-60 minutes. Sufficient precipitation will be evidenced by the formation of a viscous precipitate consisting of agglomerized cells and insoluble proteins.

The mixture is then centrifuged for about 5-30 minutes at 1,000×g to pack the precipitate at the bottom of the tube. Finally, the supernatant above the precipitate is removed from the tube; the supernatant being that fraction of the mixture that contains purified autologous thrombin.

In one embodiment, the volume of whole blood used to prepare the autologous thrombin will be small, for example, as little as 8 to 10 ml. The blood is drawn into a blood collection tube (e.g. a VACUTAINER® tube) or syringe containing a non-heparin anticoagulant. Examples of anticoagulants that may be used in the invention include calcium ion-binding or sequestering anticoagulants, such as, citrate-phosphate-dextrose (CPD) or acid-citrate-dextrose (ACD), sodium citrate, and the like. Under typical circumstances, the preferred anticoagulants are acid-citrate-dextrose (ACD) and ACD/mannitol.

Typical precipitating agents will include, for example, polyethylene glycol, ammonium sulfate or ethanol, as well as such components as calcium chloride or magnesium chloride.

In one embodiment, ethanol is used as a precipitating agent. The final concentration of ethanol will preferably be between 10% and 25%. For an 8 to 10 ml starting whole blood volume, therefore, 1 to 2 ml of 100% or 95% ethanol is added to the whole blood.

Additionally between about 0.05 and 0.4 ml of a 10% solution of calcium chloride is added to the mixture of anticoagulated whole blood and precipitating agent. For example, in one embodiment, with a starting anticoagulated whole blood volume of 8 ml, a mixture of 1.6 ml ethanol and 0.1 ml of 10% CaCl₂ was used.

With respect to the time sufficient for precipitation of the cellular and specific plasma components to occur, precipitate may be expected to form in the tube within about 5 to 45 minutes.

In one embodiment, the initial volume of whole blood may be anticoagulated with a mixture of ACD and mannitol, with the concentration of mannitol being about 5-10 mg/l ml ACD.

Preparation of Bone Graft Material

In one embodiment of the present invention, the bone marrow concentrate (BMC) is then combined with purified autologous thrombin (AT) in a BMC:AT ratio sufficient to promote clotting of the bone marrow concentrate, preferably 1:1-6:1, and more preferably, 3:1-5:1. Clotting of the BMC may be timed to occur prior to or following insertion of the graft materials into the graft site.

Composite bone graft materials frequently include a porous implantable matrix which provides a scaffolding for the distribution of bone-healing progenitor cells and growth factors. While no matrix is required for the bone graft material of the present invention, if in the surgeon's discretion, a matrix material is desired for the particular indication, the coagulated bone marrow concentrate may be combined with matrices conventionally used to facilitate bone fusion, for example, mineralized and demineralized cancellous bone, and various synthetic matrices including coralline hydroxyapatite.

Preparation of Platelet Rich Plasma (PRP) or Platelet Concentrate (PC)

Platelet rich plasma (PRP) and/or a platelet concentrate (PC) may also be combined with the bone marrow concentrate prior to exposure with purified autologous thrombin to form the bone graft composition of the invention.

In one embodiment of the invention, separation and concentration of bone marrow and peripheral blood platelets and white blood cells is achieved by combining an effective amount of bone marrow aspirate and peripheral blood and processing this composition as described for concentration of bone marrow separately.

The combination of purified autologous thrombin with the anticoagulated bone marrow concentrate results in the formation of a coagulated tissue graft composition. Timing of activation of the bone marrow concentrate to form the coagulated tissue graft composition of the present invention varies. In one embodiment, the bone marrow concentrate may be injected into the graft site prior to or simultaneously with addition of purified autologous thrombin so that clotting of the bone marrow concentrate occurs in situ. Alternatively, the bone marrow concentrate and purified autologous thrombin may be combined ex vivo and applied directly to the graft site in a coagulated form. In yet another embodiment, supplemental graft material may be combined with the bone marrow concentrate prior to or subsequent to coagulation with purified autologous thrombin.

A canine critical bone defect model is used to illustrate the healing ability of a novel mixture of bone marrow-derived cells, autologous thrombin and optimally, platelet concentrate. The critical bone defect is produced by resection of a 21-mm diaphyseal section of the femur that results in a non-union if left untreated.

Primary outcome assessment is the load-bearing function of the regenerated tissue. Bone healing is followed serially with radiographs. Since radiographic scoring correlates poorly with mechanical strength and stiffness, however, bone fusion in the defect is tested mechanically in torsion to failure 16 weeks after surgery.

### EXAMPLE 1

Forty purpose-bred, skeletally mature cross breed hounds of both sexes weighing about 25 kg are divided into four study groups: A) autologous bone graft harvested from the ipsilateral ilium; B) Autologous platelet concentrate (PC) and autologous bone marrow concentrate (BMC; C) PC, BMC and autologous thrombin (AT); D) PC, bone marrow aspirate (BMA) and AT. A duplicate study includes a supplemental graft material, such as tricalcium phosphate (TCP) matrix in groups B-D.

A 21 mm defect is surgically created at the middiaphysis of the femur, stabilized with a plate and treated. The defect is allowed to heal for 16 weeks. Animals are sacrified and the quality of the bone is analyzed using radiographic and biomechanical techniques.

Surgical Procedure

The surgical procedure is described by Kraus et al. and is summarized here. The femur to be operated is chosen in a controlled randomized fashion and prepared for aseptic surgery. A standard lateral approach is made to the femur. An 8-hole, 135 mm long, 4.5 mm, leg-lengthening plate (available from Synthes, Paoli, Pa.) is contoured and applied with bicortical screws to the lateral aspect of the femur. The plate is removed and a 21-mm cylindrical section of diaphysis and its associated periosteum is removed with an oscillating bone saw. The surgical site is copiously lavaged during the osteotomy procedure to avoid heat necrosis of the bone and to remove all bone debris. The plate is reapplied. The defect is filled with the appropriate graft material according to the treatment randomization.

Autologous Cancellous Bone Graft Procedure (Control Group)

Autologous cancellous bone is harvested from the greater tubercle of the humerus ipsilateral to the femur to be operated. The humerus is prepared for aseptic surgery. A 12-mL syringe is prepared preoperatively by cutting off the end where a hypodermic needle attaches. The inner diameter of the 12 mL syringe is 14 mm, similar to the mean diameter of the femoral diaphysis in these dogs. The autologous cancellous bone chips collected with a 7 mm curette is immediately transferred to the 12-mL syringe. The syringe is filled to 21 mm (the length of the defect in the diaphysis). The cancellous bone graft will partially clot during collection forming a cylinder with dimensions 14 mm wide and 21 mm long. The cylindrical graft is transferred to the defect from the syringe. A periosteal elevator is used to mold the graft to completely fill the cavity. Wound closure is routine. The dogs are returned to separate runs after recovery from surgery and an overnight stay in the intensive care unit.

Preparation of Platelet Concentrate (PC) and Autologous Thrombin (AT)

After induction of anesthesia, the cervical area is aseptically prepared and jugular venipuncture is performed with a 19 gauge needle and a total of 59 ml of whole blood is collected. 45 ml of whole blood is collected in the first syringe containing 5 ml of an anticoagulant (anticoagulant citrate dextrose, ACD). 9 ml of blood is collected in a second syringe containing 1 ml of an anticoagulant (mannitol and anticoagulant citrate dextrose, ACD). The 50 ml of blood/ACD mixture is centrifuged using the SMARTPREP® system for separation of the blood into PC. This process will yield a PC volume of 5 ml. From the total PC volume, 1 ml is used for PC assay and 4 ml is placed in a sterile container for mixing with BMC and mixing with AT. The 10 ml blood/mannitol ACD mixture is centrifuged in the SMARTPREP® system and 4 ml of AT is produced.

Preparation of Bone Marrow Concentrate (BMC)

After induction of general anesthesia, the proximal humerus, ipsilateral to the femur that is to be operated, is prepared for aseptic surgery as described above. Twelve, 2 ml aspirates are collected from the proximal humerus separated by approximately 1 cm by changing the direction and depth of the needle placement. Two such aspirates are drawn into a 10 ml syringe containing 2 ml of ACD as an anticoagulant. This process is repeated to obtain three syringes from the left humerus and three from the right. The total bone marrow volume drawn is 24 ml. The total anticoagulated volume is combined in a sterile cup for a total pooled sample volume of 36 ml. A 1 ml sample of the pooled sample is used to assay the number of nucleated cells present as a baseline. The remaining 35 ml volume is placed in a separation disposable cartridge and centrifuged using the SMARTPREP® system for separation of the components of the bone marrow aspirate. This process will yield 4 ml of bone marrow concentrate which is placed in a sterile cup to be used in preparing the implantation graft material in Groups B or C.

Preparation of Bone Marrow Aspirate (BMA)

Following the same collection protocol as for preparation of the bone marrow concentrate, a bone marrow aspiration needle is inserted into the greater tubercle of the proximal humerus. Two aspirates of 2 ml each are drawn, one from the left humerus and one from the right. Each syringe contains ACD anticoagulant. The contents of both syringes are used in Group D and may be combined with the TCP matrix.

Preparation of Grafts for Surgical Implantation

Group B implants are prepared as follows: 4 cc of TCP is rehydrated in 4 ml of BMC. The TCP material is placed into a 5 ml syringe and 4 ml of BMC is delivered into the graft material. After 120 seconds, 1.5 ml of AT is delivered into the TCP/BMC material. A 3 ml syringe with a cannula is used to deliver the AT. After delivery, this mixture is allowed to set up for 120 seconds. The resultant formed graft material is expressed from the syringe and placed into the defect.

Group C implants are prepared as follows: 4 cc of TCP is rehydrated in 4 ml of BMC. The TCP material is placed into a 5 ml syringe and 4 ml BMC is delivered into the graft material. This hydrated material is allowed to stand for 120 seconds. Then a 2 ml mixture of PC and AT is delivered in a 3:1 ratio. The delivery of these combined autologous materials is accomplished through a dual lumen cannula pushed through the center of the TCP/BMC material to the base of the syringe. The PC/AT mixture is expressed simultaneously as the cannula is withdrawn from the syringe containing the TCP/BMC. This is a quick and easy method to achieve a uniform distribution of the autologous materials throughout the graft material. After delivery, this mixture is allowed to set up for 60 seconds. The resultant formed graft material is placed into the defect. The surface to the graft material and the surrounding tissue is then coated with 2.5 ml PC and 0.7 ml AT expressed simultaneously from a dual lumen cannula to seal the surface of the graft and place an enhanced level of proteins on the adjacent tissue.

Group D implants are prepared as follows: 4 cc of TCP is placed into a 5 ml syringe. 4 ml of BMA is added to the TCP material and allowed to stand for 120 seconds. Then a 2 ml mixture of PC and AT is delivered in a 3:1 ratio. The delivery of these combined autologous materials is accomplished through a dual lumen cannula pushed through the center of the TCP/BMC material to the base of the syringe. The PC/AT mixture is expressed simultaneously as the cannula is withdrawn from the syringe containing the TCP/BMC. After delivery, this mixture is allowed to set up for 120 seconds. The resultant formed graft material is expressed from the syringe and placed into the defect. The surface to the graft material and the surrounding tissue is then coated with 2.5 ml PC and 0.7 ml AT expressed simultaneously from a dual lumen cannula to seal the surface of the graft and place an enhanced level of proteins on the adjacent tissue.

Clinical Examination

Body temperature, pulse, and respiration rate is monitored daily. Dogs are evaluated for lameness daily on a 5 point scale (Budsberg, et al. 1993)⁶⁴. The femoral diaphysis is palpated daily after one week and the response to deep palpation is recorded.

Radiography

All dogs are sedated with acepromazine (0.2 mg/kg) and butorphanol (0.2 mg/kg) intravenously for chemical restraint during radiography. Preoperatively, standard cranial-caudal (anterior-posterior) and lateral radiographs of both femora are taken to ensure that all dogs have normal hind limbs. Postoperative cranial-caudal and lateral radiographs are taken to verify implant placement and to serve as a baseline for radiographic evaluation. Serial cranial-caudal radiographs of the operated femur only is taken at 4, 8, 12, and 16 weeks as the leg-lengthening plate used to stabilize the fracture will obscure the bone healing defect in the lateral projection. At 16 weeks postoperatively, the lateral radiograph is taken after the plate and screws are removed.

The same radiographic technique (voltage and time) is used at each session. A radiographic standard is placed at the level of the femur that consists of two radiopaque spheres spaced 100 mm apart. This standard will allow for correction of magnification and reliable measurement of the femoral diameter. An aluminum step wedge phantom will also be included for density quantification. A radiologist will evaluate the radiographs for evidence of bone healing according to established criteria. Scores are based on mineralization, bony bridging, continuity of bony bridging, loss of cortex at each end of the defect as bone forms, size of extraosseous callus, and integrity of the bone-plate construct, especially signs of screw loosening. The radiologist is blinded to treatment-group assignment.

Necropsy Evaluation

Exactly 16 weeks after surgery, the dogs are euthanized with an overdose of barbiturate and the femurs harvested for testing. High-resolution radiographs and computer tomography scans are made of the excised tissue at the time of euthanasia. The vastus lateralis and biceps muscles are carefully dissected from the callus. Photographs are taken with a standard rule in the plane of the photograph. Calipers are used to measure the thickness of the callus as seen radiographically on the medical aspect of the implant at three locations: the proimal and distal host cortex-implant interfaces and the middle portion of the implant.

Computed Tomography (CT)

Computed tomography (CT) scans are taken of the excised femora (Picker PQS CT system). A Plexiglass fixture is used to consistently position the bones. 2-mm thick image slices are acquired at 1-mm spacing, producing 1 mm of overlap. A Cann-Genant bone phantom is included in the field of view to allow conversion of the CT (Hounsfield) units to tissue mineral density.

A 3-cm high volume of interest (VOI) is reconstructed including the defect. The 21-mm defect is selected within the VOI for analysis. The bone mineral density (g/cm³) histogram is determined slice by slice along the defect and for the entire graft VOI by summing across the graft slices. The mineralized graft volume is also determined. If sufficient remodeling has occurred, the polar moment of inertia and section modulus of the newly formed bone tissue is calculated from the CT reconstructions and correlated with the torsional parameters from strength testing.

Biomechanical Testing

The femora is wrapped in saline-soaked gauze, double bagged, and stored at −20 C until mechanical testing. The whole bone strength of both operated and contralateral non-operated femurs is tested. Only specimens radiographically graded as healed is tested.

Whole bone stiffness and strength is determined by a failure torsion test using a previously published protocol⁶⁸. The proximal and distal ends of each femur is removed to create a prismatic cylindrical specimen, leaving 2-3 cm on each side of the 21-mm defect. Then the cut ends of the femur are embedded in square aluminum tubing with fast setting polymethylmethacrylate (COE Tray Plastic, Fast Set). A 3-cm spacer is used to maintain a consistent gauge length across the defect in all specimens. The two ends are aligned using a custom fixture constructed from aluminum channel. The gauge segment of the contralateral control is matched to correspond with the operated femur. The bones are maintained moist during preparation with phosphate buffered saline.

The prepared specimens are tested in torsion using a servohydraulic biaxial load frame (MiniBionix 858, MTS Systems). The torque is applied at 1 degree/second failure, and no axial load is applied. Torque and angle is sampled at 5 Hz throughout the test. The angular displacement normalized by the gauge length is the twist. The torsional stiffness is determined from the initial linear portion of the torque-twist curve. The torsional strength is the torque at which failure occurred, and the corresponding deformation is the twist at failure. The energy absorbed to failure is calculated from the area beneath the torque-twist curve.

Thus, the present invention provides a method of preparing an autologous tissue graft material having the following characteristics:

1. It can be prepared at point of care from a bone marrow aspirate and a whole blood sample from the individual receiving the graft during the course of a surgical procedure to implant the graft.

2. The autologous bone marrow graft material can be delivered to the graft site alone or in conjunction with a platelet concentrate or platelet rich plasma by a variety of techniques or devices.

3. The autologous bone marrow graft material of the present invention can be applied directly to a graft site or combined with another graft material.

1. A method for the preparation of a tissue graft material comprising:
a) obtaining a volume of anticoagulated bone marrow from a patient;
b) obtaining a concentrate of said bone marrow; and
c) mixing the bone marrow concentrate with purified autologous thrombin to obtain the tissue graft material.

2. The method of claim 1, wherein said concentrate of nucleated cells is obtained by centrifugation of the bone marrow at or near point-of-care.

3. The method of claim 1, wherein the bone marrow is anticoagulated with an anticoagulant selected from the group consisting of citrate-based anticoagulants, heparin and EDTA.

4. The method of claim 1, further comprising the step of combining said bone marrow concentrate with a platelet concentrate prior to step c).

5. The method of claim 1, further comprising the step of combining said bone marrow concentrate with a supplemental graft material prior to step c).

6. The method of claim 1, further comprising the step of combining said bone marrow concentrate with a supplemental graft material subsequent to step c).

7. The method of claim 4, further comprising the step of combining said bone marrow concentrate and platelet concentrate with a supplemental graft material prior to step c).

8. The method of claim 4, further comprising the step of combining said bone marrow concentrate and platelet concentrate with a supplemental graft material subsequent to step c).

9. A method for the preparation of a tissue graft material comprising:
a) obtaining a volume of anticoagulated bone marrow from a patient;
b) mixing the bone marrow with purified autologous thrombin to obtain the tissue graft material.

10. The method of claim 9, wherein the bone marrow is anticoagulated with an anticoagulant selected from the group consisting of citrate-based anticoagulants, heparin and EDTA.

11. The method of claim 9, further comprising the step of combining said bone marrow concentrate with a platelet concentrate prior to step c).

12. The method of claim 9, further comprising the step of combining said bone marrow concentrate with a supplemental graft material prior to step c).

13. The method of claim 9, further comprising the step of combining said bone marrow concentrate with a supplemental graft material subsequent to step c).

14. The method of claim 11, further comprising the step of combining said bone marrow concentrate and platelet concentrate with a supplemental graft material prior to step c).

15. The method of claim 11, further comprising the step of combining said bone marrow concentrate and platelet concentrate with a supplemental graft material subsequent to step c).

16. A method for the preparation of a tissue graft material comprising:
a) obtaining a volume of anticoagulated bone marrow from a patient;
b) obtaining a concentrate of said bone marrow;
c) obtaining a volume of anticoagulated whole blood from the patient;
d) mixing said anticoagulated whole blood with a precipitating agent;
e) incubating the mixture of step d) for a time sufficient for precipitation of cellular and specific plasma components to occur;
f) separating the precipitant obtained from step e) to obtain a supernatant wherein said supernatant contains purified autologous thrombin;
g) mixing the bone marrow concentrate of step b) with purified autologous thrombin of step f) to obtain the graft material.

17. The method of claim 16 wherein steps c) through f) are performed concurrently with steps a) through b).

18. The method of claim 16 wherein steps c) through f) are performed prior to steps a) through b).

19. The method of claim 16, wherein said bone marrow concentrate is obtained by centrifugation of the bone marrow.

20. The method of claim 16, wherein the bone marrow is anticoagulated with an anticoagulant selected from the group consisting of ACD, CPD, heparin and EDTA and other suitable anticoagulants.

21. The method of claim 16, further comprising the step of combining said bone marrow concentrate with a platelet concentrate prior to step g).

22. The method of claim 16, further comprising the step of combining said bone marrow concentrate with a supplemental graft material prior to step g).

23. The method of claim 16, further comprising the step of combining said bone marrow concentrate with a supplemental graft material subsequent to step g).

24. The method of claim 21, further comprising the step of combining said bone marrow concentrate and platelet concentrate with a supplemental graft material prior to step g).

25. The method of claim 21, further comprising the step of combining said bone marrow concentrate and platelet concentrate with a supplemental graft material subsequent to step g).

26. A tissue graft composition comprising autologous bone marrow and purified autologous thrombin.

27. The composition of claim 26 wherein said composition further comprises at least one graft material selected from autograft, allograft, xenograft and a synthetic graft material.

28. The composition of claim 26 wherein said composition further comprises a platelet concentrate.

29. The composition of claim 28 wherein said composition further comprises at least one bone graft material selected from autograft, allograft, xenograft and a synthetic graft material.

30. A tissue graft composition comprising a bone marrow concentrate and purified autologous thrombin.

31. The composition of claim 30 wherein said composition further comprises a graft material selected from autograft, allograft, xenograft and a synthetic bone graft material.

32. The composition of claim 30 wherein said composition further comprises a platelet concentrate.

33. The composition of claim 32 wherein said composition further comprises at least one graft material selected from autograft, allograft, xenograft and a synthetic graft material.
